(19) United States
(12) Patent Application Publication
(10) Pub. No.: US 2009/0176871 A1
(43) Pub. Date: Jul. 9, 2009

(54) Treatments for Domestic Animals Having Sex Hormone Deficiencies Using Soy Germ Isoflavones

(76) Inventor: Phillip E. Schoenwetter, Rancho Palos Verdes, CA (US)

Correspondence Address: EDWARD C. SCHEWE, 1600 Rosecrans Avenue, Fourth Floor, MANHATTAN BEACH, CA, 90266, US

(21) Appl. No.: 12/349,727

(22) Filed: Jan. 7, 2009

Related U.S. Application Data
(60) Provisional application No. 61/010,234, filed on Jan. 7, 2008.

(51) Int. Cl. A61K 31/352; C07D 311/36

(52) U.S. Cl. 514/456; 549/403

(57) ABSTRACT

The novel applicability and methods of use of soy germ extracted isoflavones for the treatment of sex hormone loss due to spaying, neutering and aging in domestic pets as well as service and guide animals.

### RELATED APPLICATION DATA

This application claims the benefit of U.S. Provisional Application Ser. No. 61/010,234 filed Jan. 7, 2008 entitled Treatments for Domestic Animals Having Sex Hormone Deficiencies Using Soy Germ Isoflavones.

### BACKGROUND OF THE INVENTION

1. Field of the Invention

The field of the invention is treatment of sex hormone deficiencies in domestic animals.

2. Description of the Related Art

For over four decades the cornerstone of the humane treatment of domestic pet animals has been animal population control. This is universally accomplished by spaying and neutering which removes the ovaries and testes. Although 100% effective, gonad removal also removes most all the sex hormones.

The adverse effects of sex hormone loss in men and women are well recognized and much effort is given to hormone replacement and/or other off setting treatments. Veterinary science is also aware of the adverse physiologic impact of sex hormone loss or deficiency in animals but has not advanced either a prescription or non prescription remedy with an excellent and well known safety profile. A need exists for a remedy for the adverse physiologic impact of sex hormone loss or deficiency in animals, especially those spayed or neutered at an early age.

### SUMMARY OF THE INVENTION

The inventor herewith has discovered a novel applicability of soy germ extracted isoflavones for the treatment of sex hormone loss due to spaying, neutering and aging in domestic pets as well as service and guide animals. Sex hormones work by a number of different metabolic pathways and this is a reason why so many organ systems in addition to the reproductive system are impacted by sex hormone loss.

Sex hormone loss affects the genitourinary system by atrophy of the uro-genitalia and especially lessens tension of the trigone muscle leading to more frequent urinary tract infections, less bladder control and increased urinary leakage. This can produce an unpleasant odor about the pet as well.

Sex hormone loss clearly is the principal cause of medullary and cortical bone thinning in both axial and non axial bone. The demineralization of bone is responsible for the subchondral (the bone just under the joint cartilage) bone plate weakness which is the most common cause of degenerative arthritis in domestic pets. There is presently a large amount of money being spent on veterinary consultations, diagnostic studies and medicines as well as needless pain and immobility endured by our pets when this can readily be prevented or limited in most cases by using the present invention.

Additionally, the attachments of ligaments and tendons to bone are also weakened with significantly much more such injuries in spay/neuter animals than natural animals of the same age. Further, arthralgias limit mobility and physical activity which has other cardiovascular consequences as well. Demineralization of the alveolar and mandibular bone is a leading cause of loosening of the teeth in their sockets and a prime cause of periodontal disease in spay/neuter animals with over a two-fold rate of tooth loss compared to natural animals.

Optimal body weight has health implications in animals as it does with people. Natural animals have much more frequently and more easily attained ideal body weights than their spayed and neutered counterparts. Sex hormones play an important part in the satiation feedback loop. Natural animals will tend to quit eating and not overeat as much as those that, are spayed and neutered. Being overweight in animals promotes hypertension and excessive joint stress as well as adverse blood lipid profiles. Hypertension is thought to be the leading cause of liver and kidney disease in dogs.

Sex hormones foster secretion of nerve growth factors that stimulate new neuron growth in the brain as well as foster maintenance and preservation of cognitive function in animals. Sex hormones play a supportive role in maintaining retinal and eye lens thickness.

In embodiments of the invention, the particular isoflavones extracted from soy germ are daidzein and genestein. The metabolite of daidzein, Equol, has similar effectiveness as daidzein and genestein. The principal mechanism of action of these isoflavones is that they function as estrogen beta agonists. That is, the isoflavones work by attaching to the Estrogen Receptor Beta (ERB) and stimulating the same cell activity that the natural hormone would do to any cell that has an ERB receptor.

The receptors have different names because, although they get activated by the very same hormone or chemical, the bottom line effect can be drastically different. An example with something most individuals have experience with is histamine and the “antihistamine” drugs we take to ward off running noses, itchy eyes, sneezing and itchy skin and rashes etc. When we take an “antihistamine” for hay fever, we are really taking what is technically a Histamine 1 Blocker. When histamine reacts with the H1Receptor it initiates the cascade that we end up perceiving as itch, sneezing, running nose etc. Common medications of this sort are Bendryl, Drixoral, Chlortrimeton, Zyret etc. However, if that same histamine were to react with a Histamine 2 Receptor, which the stomach has plenty of, it would cause the secretion of lost of gastric acid and promote gastric and duodenal erosions and ulcers.

The “antihistamines” that are really H2 blockers that we may have experienced are Tagamet, Axid, Zantac, etc. So the very same histamine, if reacting with a H1 Receptor causes itch and sneezing, can cause ulcers if attaching to an H2 Blocker. This same duality exists for estrogen as there is known to be both alpha and beta receptors that have diverse and at times, opposite effects when stimulated.

### DETAILED DESCRIPTION OF EMBODIMENTS OF THE INVENTION

In one embodiment, the invention is a consumable biscuit made principally from chicken, barley, oats and rice as well as isoflavones (“ISOS”) and flavorings, bone meal, vitamins and preservatives. The biscuit is uniquely produced from roasted chicken, malted barley flour, rice flour, steel cut oats, and malt, hulled barley grain, bone meal, flavorings, vitamins and minerals and anti-oxidants/preservatives.

A novel preparation of the biscuit relates to a method of chicken preparation and how it and the bone meal is made into a dough-like pate' texture that blends seamlessly with the grains and malted flour and rice flour with the steel cut oats and hulled barley providing a biscuit baked feel, shelf life and appearance although the overall moist weight of the dough is 43% roasted chicken.

This novel method of the chicken preparation is achieved as follows by including the following steps:

Step One. Human grade chicken “logs” Chickens with necks and gizzards are sectioned and oven roasted and flavoring and spices added to the roasting process.

Step Two. The roasted chicken log sections are placed in a pressure cooker with chicken stock, salt and spices and then pressure cooked for 30-45 minutes depending at what altitude the process is done. In one or more embodiments, the temperature of the pressure cooker is 244° F. at mean sea level (MSL)

Step Three. The pressure cooked chicken sections including the skin and bones and remaining water in the base of the pressure cooker and placed in a food processor and blended to a totally smooth pate'.

The pressure cooking has altered the structure of the bone allowing complete fine powder like texture which allows bone and marrow to be imperceptibly incorporated into the pate' which in turn allows the pate' to seamlessly become part of the unbaked dough.

The advantages of this are multiple. First, organic bone and bone-matrix allow substantially greater calcium and phosphate absorption into the body and greater net accretion to the bone of animals that ingest this matrix compared to calcium and phosphates salts that are normally mixed with conventional animal treats.

Second, nutrients from the chicken marrow are normally not available to domestic animals because of the normal splintering tendency of normally cooked chicken bones to produce needle like edges that can easily cause choking or perforate the intestinal tract and cause bleed and infection. By being a pate', the bones have an equivalent texture of fine milled flour and is completely safe. Third, by producing a very flavorful pate that is completely and homogeneously a part of the biscuit dough that is then completely baked at high altitude, such as in Lake Tahoe, to a very low (less than 7% moisture content), users are able to make the equivalent of a dry cooked micro fine chicken jerky that has excellent shelf life, a non meat or greasy feel to the biscuit when given by the owner to the pet yet provides excellent masking of the otherwise bitter and distasteful flavor of the essential isoflavones. Another novel application is use of hulled barely and steel cut oats to provide grit for plaque and bad breath control.

There are estrogen beta receptors (ERB) in both male and female animals and that is why the benefits will accrue to males as well as females. The reader should understand that the mechanism of action on all the organ systems will stem from the ERB agonistic effects but greater specificity is presented.

### Skeletal System Claims

ISOS will maintain bone and ligament strength.
ISOS will limit and largely prevent degenerative (wear and tear) joint disease.
ISOS will lessen periodontal disease and subsequent tooth loss.
The ISOS are unique in that they one of the few substances that increase bone building activity directly. In the presence of sufficient calcium, phosphates and Vitamin D3 natural estrogens, androgens and ISOS can actually stimulate osteoblastic (bone building) cell activity.

Presently, most bone preserving medications used for humans are osteoclast (bone dissolving) inhibitors and bone preservation is attempted by limiting demineralizing processes rather than by stimulating bone building mechanisms. Weight, stress and bone healing such as from a fracture, can stimulate bone growth but these mechanisms are usually not in play on a day to day basis.

The areas of the skeleton that ISOS really shine are in joint preservation, arthritis prevention and teeth preservation. By maintaining the bone density and bone strength of the subchondral (under the cartilage joint tissue) bone plate, the ability of the chondrocytes to repair themselves in normal activities is maintained. However, with demineralization comes bone plate weakening and the subchondral floor is damaged adding to the stress of wear and tear degenerative stress of the joint. The joint chondtocytes can't repair as fast as they are injured and this chronic inflammatory condition precipitates a vicious cycle of inflammation and further joint injury.

Additionally to the factor of general “bone density” and “mineral content” is the equally important necessity of bone micro architecture. A pile of 1000 bricks won't have the same structural. load bearing capacity as 1000 bricks built as a Roman arch. This loss of architecture explains why there is over a 34% loss of bone strength and shear resistance with only a 4.5% bone mineral loss. Important to animals is the ability to eat and chew food. The incidence of tooth loss is two to threefold in spay/neutered animals due to the very high incidence of periodontal disease resulting bone loss in the mandible and alveolar bones. ISOS replete bone loss in those bones resulting in better oral health to the animals. Better teeth support better nutrition and overall health.

### Genitourinary Claims

ISOS will maintain urinary bladder control and limit hormone loss of bladder control and urinary tract infections. This will tend to limit bad odors as well. In male animals this will limit prostatism and bladder incontinence problems and urinary infections.

The trigone muscle is known to have ERB and the beneficial effects of HRT (Hormone Replacement Therapy) for females has been known for close to 50 years. ERB antiproliferative effects on the prostate have recently been realized with ISOS. The use of Estrogens in prostate cancer has been done for over 40 years. HRT has been very useful for incontinence for many years either systemically or by local cream application.

### Neurological Claims

ISOS will help maintain cognitive function and limit age related senility in spay/neutered animals. ISOS are known to stimulate nerve growth factors which in turn stimulate new neuron growth is some areas of the brain. Cognitive as well are ocular function has been enhanced or rate of degeneration delayed. ISOS have been shown to thicken the retina and help prevent macular degeneration as well as maintenance of ocular lens thickness.

### Cardiovascular Claims

ISOS promote cardiovascular health through a number of synergistic mechanisms. ISOS are known to have a blood pressure lowering effect which will limit heart problems and likelihood of heart failure. Additionally, hypertension is thought to be the main cause of “idiopathic” liver and kidney disease in animals. Additional cardiovascular benefits are garnered from the better joint protection from ISOS that encourage running and activity, sport and endurance that promotes cardiovascular health. The exercise that the animals can participate in will also enhance muscularity and lean mass to total body weight ratios. ISOS are known to lower triglycerides and LDL cholesterol and increase HDL cholesterol levels.

### Longevity and Vitality Claim

ISOS will enable animals to better fulfill their genetic code requirements for their sex hormone balance. Minimizing hormone deficiencies, maximizing organ systems restorative abilities and maintenance of normality of function of various organ systems will favorably impact longevity and the robustness of life for animals just as these factors have increased longevity and vitality in humans over the past 75 years. The maintenance of “normal” physiology will allow the maximum of life in both duration and functionality.

While the present invention has been described with regards to particular embodiments, it is recognized that additional variations of the present invention may be devised without departing from the inventive concepts described herein and includes the full breadth and scope of the claims.

What is claimed is:

1. A composition for the treatment of sex hormone deficiencies in a domestic animal resulting from spaying, neutering or aging comprising one or more soy germ isoflavones.

2. The composition of claim 1 wherein the one or more soy germ isoflavone is selected from the group consisting of daidzein and genestein.

3. The composition of claim 1 wherein the composition is administered in a form selected from the group consisting of biscuits, capsules, powders and the like.

4. The composition of claim 1 wherein the domestic animal is a dog or a cat.

5. A method for the treatment of a domestic animal to maintain bone and ligament strength in the animal by administering the composition of claim 1 to the animal.

6. A method for the treatment of a domestic animal to treat degenerative joint disease in the animal by administering the composition of claim 1 to the animal.

7. A method for the treatment of a domestic animal to lessen periodontal disease and subsequent tooth loss in the animal by administering the composition of claim 1 to the animal.

8. A method for the treatment of a domestic animal to maintain urinary bladder control in the animal by administering the composition of claim 1 to the animal.

9. A method for the treatment of a domestic animal to maintain cognitive function and limit age related senility in the animal by administering the composition of claim 1 to the animal.

10. A method for the treatment of a domestic animal to promote cardiovascular health in the animal by administering the composition of claim 1 to the animal.

11. A method for the treatment of high blood pressure in a domestic animal by administering the composition of claim 1 to the animal.

12. A method for the treatment of a domestic animal to stimulate neuron growth factors in the brain of the animal by administering the composition of claim 1 to the animal.

13. A method for the treatment of a domestic animal to enhance and preserve cognitive function in the animal by administering the composition of claim 1 to the animal.

14. A method for the treatment of a domestic animal to thicken the lens and retina of the eyes of the animal by administering the composition of claim 1 to the animal.

15. A method for the treatment of a domestic animal to limit macular degeneration in the eyes of the animal by administering the composition of claim 1 to the animal.

16. A method for the treatment of a domestic animal to increase longevity of the animal by administering the composition of claim 1 to the animal.

17. The method of claim 5 including the step of administering a therapeutically effective amount of the composition to the animal.

18. The biscuit of claim 3 further comprising one or more of roasted chicken, malted barley flour, rice flour, steel cut oats, and malt, hulled barley grain, bone meal, flavorings, vitamins and minerals, anti-oxidants and preservatives.
